(19) United States
(12) Patent Application Publication
(10) Pub. No.: US 2021/0113073 A1
(43) Pub. Date: Apr. 22, 2021

(54) LARGE MASS REMOVAL ENDOSCOPE APPARATUS

(71) Applicants: Sharon A. Hibbs, Overland Park, KS (US); Michael McGee, Kansas City, MO (US)

(72) Inventors: Sharon A. Hibbs, Overland Park, KS (US); Michael McGee, Kansas City, MO (US)

(21) Appl. No.: 17/077,462

(22) Filed: Oct. 22, 2020

Related U.S. Application Data
(60) Provisional application No. 62/924,266, filed on Oct. 22, 2019.

(51) Int. Cl. A61B 1/015; A61B 1/00; A61B 17/22; A61B 1/273

(52) U.S. Cl. CPC A61B 1/015; A61B 1/00135; A61B 17/22; A61B 2017/22079; A61B 1/2736; A61B 2217/007; A61B 2217/005

(57) ABSTRACT

An instrument for use in the gastrointestinal tract of a patient comprises an endoscope, a vacuum tube independently reciprocal relative to the endoscope and having a cross section of at least 18 french, and a sleeve that surrounds most of both the endoscope and vacuum tube.

### CROSS-REFERENCE TO RELATED APPLICATIONS

This application claims the benefit of U.S. Provisional Application Ser. No. 62/924,266, filed Oct. 22, 2019, the disclosure of which is hereby incorporated herein in its entirety by reference.

### BACKROUND OF THE INVENTION

The invention is to the combination of a relatively large suction tube with a conventional endoscope within a sleeve to provide for the removal of large masses, especially clotted blood, from the stomach or other regions of the body.

Doctors often encounter situations within a patient where large amounts of blood have coagulated in clumps, or other large debris is located in the stomach or elsewhere, that make it difficult for the doctor to visualize the area through a conventional endoscope and perform medical activity to correct the cause of the bleeding or to see and correct other issues producing a medical problem.

Endoscopes for upper and lower gastrointestinal tracks are frequently equipped with a light source, a water spray, an optical viewing system and a vacuum system. Unfortunately, the vacuum system associated with the conventional endoscopes is only suitable for removal of small volumes of liquid or of small particles. The large clumps, especially found with significant bleeding, cannot be removed by the comparatively narrow vacuum tube found in conventional endoscopes.

### SUMMARY OF THE INVENTION

An instrument comprising an endoscope that is joined with a comparatively large vacuum lumen or tube within a common sleeve. The overall instrument is sized to be received within narrow openings in the gastrointestinal track of the body, especially through the esophageal opening.

The vacuum tube is slideably mounted adjacent the endoscope and either the endoscope or the vacuum tube can be advanced in front of the other or together, so as to make the front end thereof more available for use. That is, the tube and endoscope are reciprocal with respect to each other and to the sleeve. A stop is provided to limit how far the vacuum tube can advance relative to the endoscope and sleeve. Preferably, the vacuum tube is in the range of 18 to 34 french and especially in the range from 24 to 28 french.

Various objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a partial side elevational view of an instrument for insertion into the gastrointestinal tract, showing an endoscope and a vacuum tube within a sleeve, with portions removed to show detail thereof and in a tube forward configuration.

FIG. 2 is a partial side elevational view of the instrument with portions removed to show detail thereof and in a tube rearward configuration.

FIG. 3 is a cross sectional view of the instrument, taken along lines 3-3 of FIG. 2.

FIG. 4 is a partially schematic prospective view of the instrument shown inserted in a patient with the tube and a gastrointestinal tract of the patient illustrated as positioned in a body of the patient and with controls for the instrument shown schematically.

### DETAILED DESCRIPTION OF THE DISCLOSURE

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The reference numeral 1 generally represents an assembly or instrument for medical use to conduct medical procedures along the gastrointestinal tract 3 of a patient 4.

The instrument 1 includes an endoscope 8 which in the illustrated embodiment is a pediatric endoscope, as such scopes are of a smaller size than adult scopes and thus function well with the present invention which includes a vacuum tube 9 and a sleeve 10. It is foreseen that the endoscope 8 could be either an upper gastrointestinal tract endoscope or a lower gastrointestinal tract endoscope or any similar scope, but it is preferred that the endoscope be a pediatric upper gastrointestinal tract scope, especially having a length of approximately 53 centimeters, because this is the length of the normal upper human gastrointestinal tract, although length can be varied with use or person to be used upon.

The endoscope 8 includes an elongate outer sheath 14 encompassing internal channels and electrical conduct 16. Endoscopes vary somewhat in what each include and the size, shape, and layout of the various channels and conducts incorporated therein and it is foreseen that virtually any such endoscope could be used in conjunction with the invention. Typical endoscopes can be seen in thousands of patents, such as U.S. Pat. No. 8,016,753, incorporated herein by reference.

The endoscope 8 illustrated has a body 19 that includes a light, air tube, and multiple water spray tubes, each joined to a light source 20, an air source 21, a water spray source 22, which provides a light spray, and an irrigation source 23 that provides a heavier flow respectively, all at a lower end or tip 26 of the endoscope 8. Such also provide optics 27 through a camera, fiber optics, or the like, that allow a physician conducting the procedure using the endoscopes 8 to view images at the tip 26 of the endoscope 8 to view what is taking place at or near the tip 26 on a screen 30, or the like. The optics are herein included in an elongate shaft 33 that can be reciprocated within the body 19 that receives the shaft 33. The various sources for light 20, air 21, water 22 and 23 are all centrally controlled through a console 36 that has conventional keyboard control, or the like, and the monitor 30.

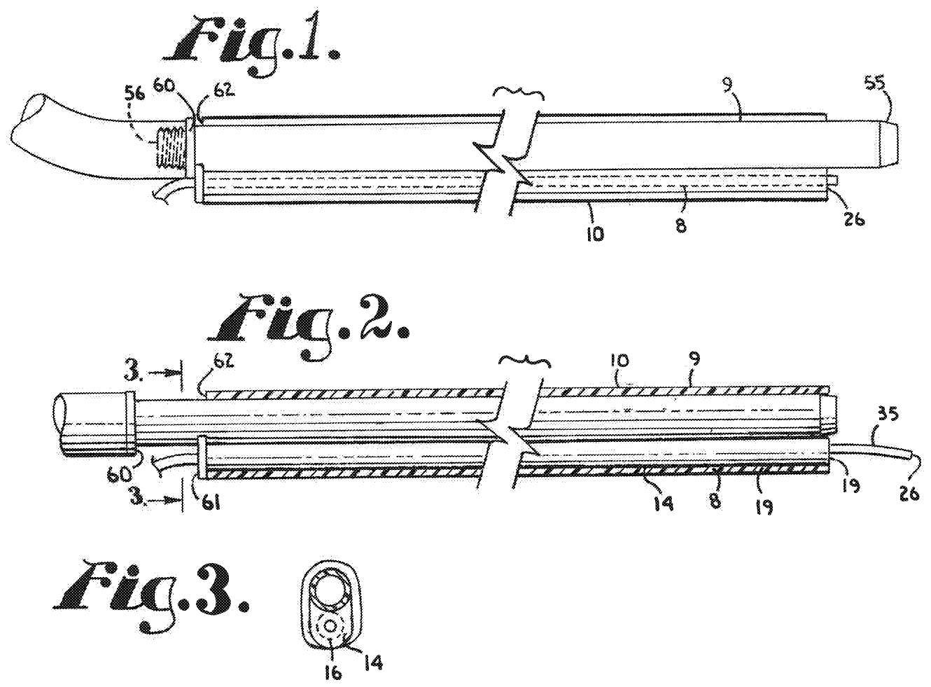

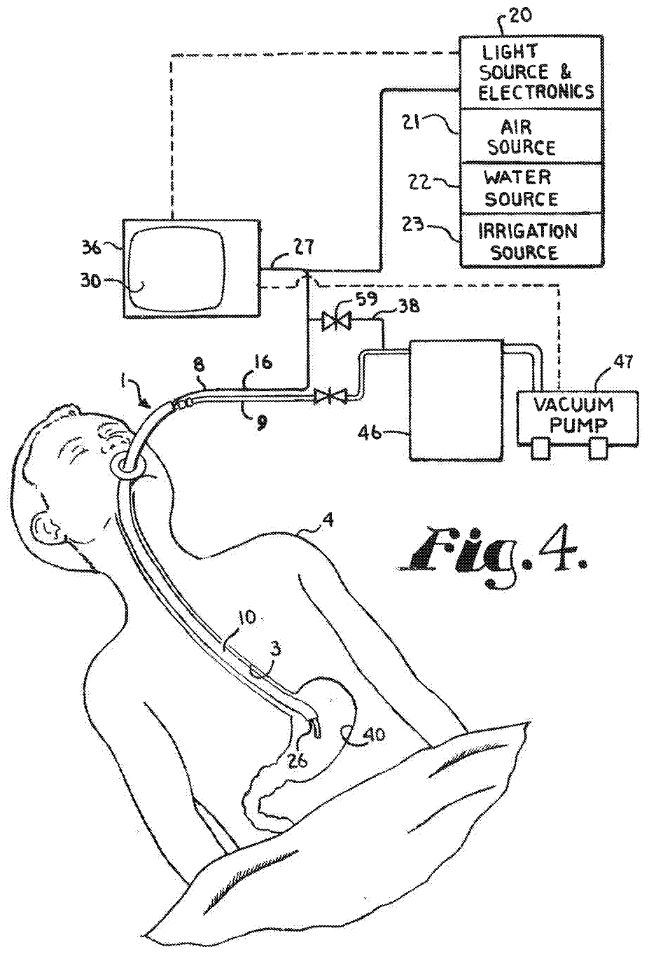

The endoscope 8 also typically incorporates a vacuum lumen 38 that is relatively narrow and typically about 4 to 6 french in size. The lumen 38 with an opening at or near the tip 26 and that is designed to remove water from sources 22 and 23, as well as other light fluids and possibly small solids from the gastrointestinal area, especially the stomach 40. The vacuum lumen 38 discharges into a collection chamber 46 which in turn is fluidly connected to a vacuum pump 47 which provides the vacuum to the lumen 38. The pump 47 is controlled by the operator through the console 36. As can be seen in FIG. 1, the endoscope shaft 33 can be retracted or withdrawn into the sleeve 10 or, as shown in FIG. 2, can be extended from the sleeve 10. Typically, the overall endoscope 8 is sized in the range of 4 to 10 french in cross section with smaller being preferred to allow a larger vacuum tube 9 to be used therewith.

The vacuum tube 9 is elongate and extends adjacent to the endoscope 8. Both the tube 9 and lower part of the endoscope 8 are generally encircled by the sleeve 10. The tube 9 is preferably about 53 centimeters in length which is the common length of the human adult upper gastrointestinal tract, but it is foreseen that it can be shorter or longer as patient size or circumstances warrant. The vacuum tube 9 is sized in cross section to approach the size of the esophageal passage when combined with the endoscope 8 and sleeve 10 to allow for the largest interior cross section of the tube 9. A preferred cross sectional size of the tube 9 is approximately 34 french; however, in some situations the size is lowered to the range of 18 to 33 french. Therefore, the range of size is typically 18 to 34 french and preferred to be 24 to 28 french. The total cross sectional size of the instrument 1 is normally maintained under 50 french to fit in the esophagus opening of most patients. This, in turn, allows removal of large debris, such as food particles, ingested non food items, and conglomerated blood. Removal of a large accumulation of conglomerated blood is the major purpose of the tube 9, but is also functions with the other items noted. Preferably, the tube has a size of 19 to 20 french, but depending on circumstances, could be less if a large endoscope must be used or more, especially if the patient has a large esophageal opening.

Located at a lower end of the tube 8 in a soft pliable sleeve 55 that is slightly tapered toward center and which acts as a barrier to prevent the tube 9 from hitting the wall of the stomach 40 or other internal structure and doing damage to the structure. The sleeve 55 is preferably about one centimeter in axial length and has approximately the same interior diameter as the tube 9 where joined thereto. The sleeve 55 is preferably constructed of a soft pliable silicone.

An upper end 56 of the tube 9 is joined to a vacuum line 57, which in turn is fluidly joined to the collection chamber 46. In this manner, when the vacuum pump 47 is on, the tube 9 can draw large pieces or clumps of debris through the sleeve 55 and eventually into the collection chamber 46. Flow through the vacuum tube 9 into the collection chamber 46 is preferably controlled by a valve 58; however, it is foreseen that a nurse or other medical practioner could simply disconnect the tube 10. A valve 59 also controls the vacuum line of the endoscope 8.

The tube upper end 56 also includes a radially extending lip 60 which is sized and positioned to engage a lip 61 on the endoscope 8 and/or the top end 62 of the sleeve 10. The tube 9 thus has a range of extension from the sleeve 10 from the configuration or position wherein it is not extended at all or withdrawn, as seen in FIG. 2, to where it is extended several centimeters, such as is seen in FIG. 1.

As can be appreciated, the tube 9 allows removal of large debris, especially large clots of blood which cannot be removed by conventional endoscopes. This allows a physician to see and treat a source of a medical problem quicker than conventional procedures.

It is to be understood that while certain forms of the present invention have been illustrated and described herein, it is not to be limited to the specific forms or arrangement of parts described and shown.

Having thus described the invention what is claimed as new and desired to be secured by Letters Patent is as follows:

1. An instrument for performing medical procedures in a patient comprising:
a) an elongate endoscope including a source of water and having an optical viewing system;
b) an elongate vacuum tube adjacent to the endoscope, the vacuum tube being at least 18 french in cross section and having a lower opening to allow debris to be drawn into the tube when under vacuum, and a vacuum system joined to the tube for drawing a vacuum therein; and
c) a sleeve surrounding both the endoscope and the vacuum tube.

2. The instrument according to claim 1 wherein:
a) the vacuum tube is circular and in the range of 18 to 34 french in cross section.

3. The instrument according to claim 1 wherein:
a) The endoscope and vacuum tube are selectively reciprocally moveable with respect to each other and the sleeve.

4. The instrument according to claim 1 wherein:
a) the vacuum tube has an upper lip that extends outwardly and engages at least one of the endoscope and an upper end of the sleeve, so as to limit downward movement of the vacuum tube relative to the endoscope and sleeve.

5. The instrument according to claim 1 wherein:
a) the vacuum tube includes a soft pliable sleeve at the lower end thereof.

6. An instrument for performing medical procedures in a patient comprising:
a) an elongate endoscope including a source of water and having an optical viewing system;
b) an elongate vacuum tube adjacent to the endoscope, the vacuum tube being 24 to 34 french in cross section and having a lower opening to allow debris to be drawn into the tube when under vacuum, and a vacuum system joined to the tube for drawing a vacuum therein; and
c) a sleeve surrounding both the endoscope and the vacuum tube.
